(19) United States
(12) Patent Application Publication
(10) Pub. No.: US 2006/0247509 A1
(43) Pub. Date: Nov. 2, 2006

(54) ECG cable for use in MRI

(76) Inventors: Mark Joseph Tuccillo, Southington, CT (US); Elliot Jay Blank, Cheshire, CT (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/414,695

(22) Filed: Apr. 28, 2006

Related U.S. Application Data
(60) Provisional application No. 60/675,951, filed on Apr. 28, 2005.

(51) Int. Cl. A61B 5/04

(52) U.S. Cl. 600/372; 600/509

(57) ABSTRACT

A cable for use in monitoring patients in an MRI environment. In one embodiment the cable is constructed of a flexible substrate on which are drawn conductive traces with a conductive ink. In one embodiment the flexible substrate is Kapton. In one embodiment the conductive ink is a carbon ink. In one embodiment the carbon ink has a resistance of 10 ohms/sq. In one embodiment the cable has a distributed impedance of 10,000 ohms/ft.

### RELATED APPLICATIONS

This application claims priority to and benefit of U.S. Provisional application 60/675,951, filed on Apr. 28, 2005, the entirety of the application is incorporated by reference herein.

### FIELD OF THE INVENTION

This invention relates to the field of monitor cables and more specifically to the field of monitor cables to be used in an MRI environment.

### BACKGROUND OF THE INVENTION

Due to recent advances in Magnet Resonance Imaging (MRI), there is a growing interest in using MRI to image the heart. Because the MRI image is very sensitive to motion from slice to slice, and stopping the heart is impractical, it is necessary to accurately detect the peak of the “R” wave of the ECG signal to generate a trigger signal and thereby insure that each image slice is taken when the heart is in the same relative position.

The traditional approach has been to use non-metallic electrodes and patient leads to bring the ECG signal out of the bore of the magnet to an ECG amplifier, where processing can occur. However, due to the nature of the MRI image acquisition, the patient is subjected to an extreme static magnetic field aligned axially to the patient; moving magnetic gradients in the X, Y, and Z axis; and pulsed radio frequency (RF) fields on the order of 1500V/meter. Each of these fields presents a special challenge for the accurate detection of the “R” wave of the ECG signal.

Considering each of these in order, the static magnetic field in most MRI devices is on the order 1.5 T (tesla) or about 5000 times stronger then the earth's magnetic field. This presents the problem that magnetic items near the bore of the magnet can become projectiles with the resulting patient or clinician injury. There is also a secondary problem with respect to “R” wave detection. Blood, which is conductive, exits the heart orthogonally to the static magnetic field. The movement of blood in the magnetic field results in a “magneto-homodynamic effect”; electrical currents induced in the blood. In other words, because blood is a conductor moving at right angles to the magnetic field, it is equivalent to a generator. The currents induced in the blood will distort the “T” wave, which indicates the re-polarization of the heart, making the “T” wave appear much larger in amplitude than it actually is. This can cause some “R” wave detection algorithms to detect the “T” wave instead, which will cause a shift of some 40 mS from the desired trigger point.

Yet a second problem is caused by the moving magnetic field gradients, since they will cause currents to be generated in any conductor exposed to them. The use of patient leads and cables inside the MRI bore to bring the low level (typically 1 mV) ECG signal to the amplifiers, results in artifacts in the ECG signal that could be in the same bandwidth as the ECG signal itself. The frequency and duration of the gradients are a function of the type of scan sequence being performed, and cannot be filtered effectively with a fixed filter sequence.

Finally, the pulsed RF fields present the greatest challenge to effective ECG detection. The RF pulse is usually a SINC ((Sin x)/x) pulse centered at 64 MHz for a 1.5 T system and is about 5 mS in duration. The repetition rate for the pulses is from tens of Hz to several KHz. The fields are generated within the magnet bore and the coils generating the pulses are excited with 50 KW of RF power creating a field strength that often exceeds 1500V/M. These RF pulses, because of the very high power, are a source of significant patient risk when the patient leads are exposed to the fields. A wire, looped around on itself, will appear as a short circuit through the insulator. This in turn will allow for the generation of eddy currents in the loop, which will then heat the wire, often enough to cause third degree burns. To limit the generation of the eddy current, the patient leads must have a distributed impedance of about 10 Kohms/ft. The result is that the RF pulses in addition to potentially generating heat in the electrodes create artifacts in the ECG amplifiers at the repetition rate used in a particular scan. The high impedance of the patient leads also increases the electrical noise of the system.

The traditional approach employed by systems presently on the market involve using carbon fiber electrodes attached to high impedance patient leads to bring the low level (1 to 5 mV) ECG signal out of the magnet bore. Once out of the bore, the high impedance leads connect to a conventional patient cable, which then supplies the signal to the ECG amplifiers. The ECG amplifiers typically are located in a RF sealed enclosure outside the magnet and often as far as 15 feet away. Because the signal is heavily contaminated with MRI related artifacts, a huge amount of post processing is required to clean the signal enough to detect the “R” wave. This process is usually DSP (Digital Signal Processing) based, and often requires the use of filters that can change parameters quickly over time to reduce artifacts. These are generally referred to as adaptive filters. Presently, no manufacturer has a solution which can produce a clean ECG waveform under all scan conditions.

The present invention helps to decrease these issues surrounding the use of a cable in an MRI field.

### SUMMARY OF THE INVENTION

The invention relates to a cable for use in monitoring patients in an MRI environment. In one embodiment, the cable is constructed of a flexible substrate on which are drawn conductive traces with a conductive ink. In one embodiment, the flexible substrate is Kapton. In one embodiment the conductive ink is a carbon ink. In one embodiment the carbon ink has a resistance of 10 ohms/sq. In one embodiment, the cable has a distributed impedance of 10,000 ohms/ft. In various embodiments disclosed herein, the cable and elements in electrical communication with it are adapted to substantially resist motion in response to a magnetic field induced by a MRI device.

In another aspect, the invention relates to a method of fabricating a cable adapted for monitoring patients in an MRI environment. The method includes the steps of providing a flexible substrate having a first surface and a second surface; and drawing a plurality of conductive traces on the first surface with a conductive ink.

In yet another aspect, the invention relates to a system for monitoring a patient in an MRI environment. The system includes a cable, an ECG monitor, and an ECG electrode. The cable includes a flexible substrate on which are drawn a plurality of conductive traces with a conductive ink, the cable adapted to substantially resist motion in response to a magnetic field induced by a MRI device. In turn, the ECG monitor is adapted to be in electrical communication with the cable. The system also includes an ECG electrode in electrical communication with the cable, the cable adapted for use in monitoring patients in an MRI environment.

### BRIEF DESCRIPTION OF THE DRAWING

FIG. 1 is a schematic diagram of an embodiment of the cable constructed in accordance with the invention.

### DESCRIPTION OF A PREFERRED EMBODIMENT

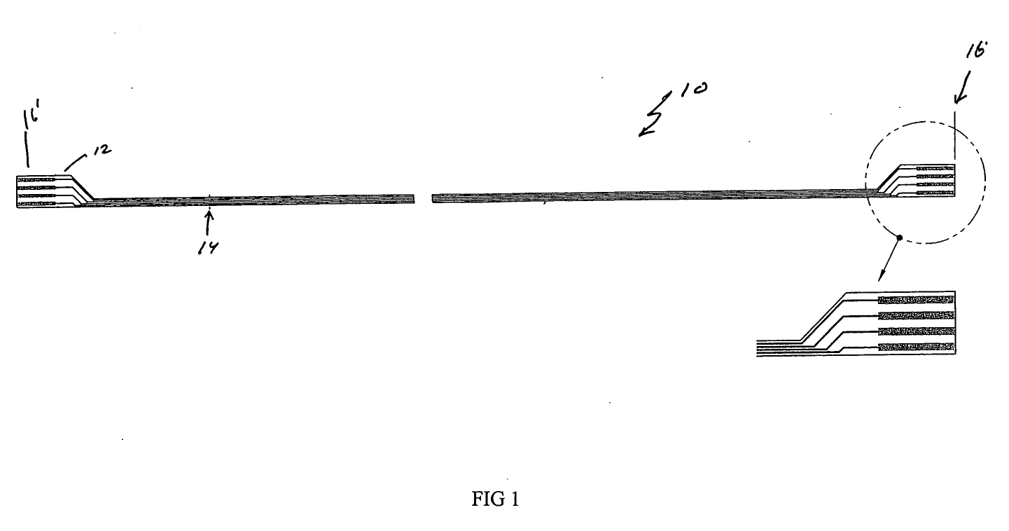

Referring to FIG. 1, a cable constructed in accordance with the invention includes a cable 10 constructed of a flexible substrate 12 on which are drawn conductive traces 14 with a conductive ink. (Ohmega Technologies, Culver City, Calif.). In one embodiment the flexible substrate is Kapton. In one embodiment the conductive ink is a carbon ink. In one embodiment, the carbon ink has a resistance of 10 ohms/sq. In one embodiment, the cable has an impedance of 10,000 ohms/ft.

In the embodiment shown the cable is a six foot long cable for use with an ECG monitor. As such the cable has four traces to conduct the signals to an ECG monitor. The two ends of the cable 16, 16′ include an expanded region with copper pads to permit one end of the cable to connect to an ECG electrode and the other end to connect to an ECG monitor.

Although the invention is described in terms of an n ECG monitor cable, the cable with the appropriate number of conductors can connect sensors on the patient with the appropriate monitor in an MRI field.

What is claimed is:

1. A cable for use in monitoring patients in an MRI environment comprising a flexible substrate on which are drawn conductive traces with a conductive ink.

2. The cable of claim 1 wherein the flexible substrate is Kapton.

3. The cable of claim 1 wherein the conductive ink is a carbon ink.

4. The cable of claim 3 wherein carbon ink has a resistance of 10 ohms/sq.

5. The cable of claim 1 wherein the cable has a distributed impedance of 10,000 ohms/ft.

6. A method of fabricating a cable adapted for monitoring patients in an MRI environment, the method comprising the steps of
providing a flexible substrate having a first surface and a second surface; and
drawing a plurality of conductive traces on the first surface with a conductive ink.

7. The method of claim 6 wherein the flexible substrate is Kapton.

8. The method of claim 6 wherein the conductive ink is a carbon ink.

9. The method of claim 8 wherein carbon ink has a resistance of 10 ohms/sq.

10. The method of claim 6 wherein the cable has a distributed impedance of 10,000 ohms/ft.

11. A system for monitoring a patient in an MRI environment, the system comprising
a cable adapted for use in monitoring patients in an MRI environment, the cable comprises a flexible substrate on which are drawn a plurality of conductive traces with a conductive ink, the cable adapted to substantially resist motion in response to a magnetic field induced by a MRI device,
an ECG monitor, the monitor adapted to be in electrical communication with the cable, and
an ECG electrode in electrical communication with the cable.

12. The system of claim 11 wherein the flexible substrate is Kapton.

13. The system of claim 11 wherein the conductive ink is a carbon ink.

14. The system of claim 13 wherein carbon ink has a resistance of 10 ohms/sq.

15. The system of claim 11 wherein the cable has a distributed impedance of 10,000 ohms/ft.
